(19) United States
(12) Patent Application Publication
(10) Pub. No.: US 2024/0212877 A1
(43) Pub. Date: Jun. 27, 2024

(54) INSULATING PLUGS, HIGH-VOLTAGE CABLES AND X-RAY TUBES

(71) Applicant: WUHAN UNITED IMAGING HEALTHCARE CO., LTD., Wuhan (CN)

(72) Inventors: Huan LIU, Wuhan (CN); Jiajia TIAN, Shanghai (CN); Shilong ZHU, Wuhan (CN)

(73) Assignee: WUHAN UNITED IMAGING HEALTHCARE CO., LTD., Wuhan (CN)

(21) Appl. No.: 18/394,508

(22) Filed: Dec. 22, 2023

(30) Foreign Application Priority Data
Dec. 22, 2022 (CN) 202211659617.5

(51) Int. Cl. H01B 7/02; H05G 1/10

(52) U.S. Cl. CPC H01B 7/0216; H05G 1/10

(57) ABSTRACT

The present disclosure provides an insulating plug, a high-voltage cable, and an X-ray tube. The insulating plug includes a first wire and an insulating layer group covering the first wire. The insulating layer group includes at least two insulating layers. The insulating layer group includes a first insulating layer covering the first wire, and a second insulating layer covering the first insulating layer. A volume resistivity of the second insulating layer is greater than a volume resistivity of the first insulating layer.

### CROSS-REFERENCE TO RELATED APPLICATIONS

This application claims priority of the Chinese Patent Application No. 202211659617.5, filed on Dec. 22, 2022, the entire contents of which are hereby incorporated by reference.

### TECHNICAL FIELD

This application relates to the field of X-ray equipment, and in particular, to an insulating plug, a high-voltage cable and an X-ray tube.

### BACKGROUND

An X-ray tube includes a shell with an internal vacuum. An emission cathode and an anode target disk are disposed in the shell. By applying negative high voltage to the emission cathode and applying positive high voltage to the anode target disk, electrons emitted from the emission cathode bombard the anode target disk under the action of an electric field, thereby generating X-rays. A metal housing is provided outside the shell, and the high voltage is introduced from the housing into the shell to apply the high voltage to the anode and the cathode. An insulating layer group is installed on the housing to isolate the high-voltage wire from the housing to avoid discharge between the high-voltage wire and the housing. However, the insulating material will age under the action of a high electric field. As the use time of the X-ray tube increases, the insulating layer group will not be able to withstand the external electric field and the insulation breakdown will occur, affecting the service life of the X-ray tube.

Therefore, it is desirable to provide an insulating plug for an X-ray tube to extend the service life of the X-ray tube.

### SUMMARY

Embodiments of the present disclosure provide an insulating plug, comprising a first wire and an insulating layer group covering the first wire, the insulating layer group including at least two insulating layers; wherein the insulating layer group includes a first insulating layer covering the first wire, and a second insulating layer covering the first insulating layer; and a volume resistivity of the second insulating layer is greater than a volume resistivity of the first insulating layer.

In some embodiments, wherein a ratio of the volume resistivity of the first insulating layer to the volume resistivity of the second insulating layer is a ratio of the radius of the first wire to the radius of the first insulating layer.

In some embodiments, wherein electric field intensities at the inner surfaces of the at least two insulating layers are equal or approximately equal.

In some embodiments, wherein the insulating layer group further includes at least one outer insulating layer covering the second insulating layer, and a volume resistivity of the at least one outer insulating layer is greater than the volume resistivity of the second insulating layer.

In some embodiments, wherein a ratio of the volume resistivity of the second insulating layer to the volume resistivity of one of the at least one outer insulating layer closest to the second insulating layer is a ratio of the radius of the first insulating layer to the radius of the second insulating layer.

In some embodiments, wherein the insulating layer group further includes at least two outer insulating layers covering the second insulating layer; and the volume resistivity of a second outer insulating layer of the at least two outer insulating layers is greater than the volume resistivity of a first outer insulating layer of the at least two outer insulating layers, the first outer insulating layer covering the second insulating layer and the second outer insulating layer covering the first outer insulating layer.

In some embodiments, wherein a ratio of the volume resistivity of the first outer insulating layer to the volume resistivity of the second outer insulating layer is a ratio of the radius of the second insulating layer to the radius of the first outer insulating layer.

In some embodiments, wherein a thickness of the insulating layer group is configured to be positively correlated with a maximum voltage carried by the first wire.

In some embodiments, wherein under a certain voltage, an average value of an electric field intensity at the insulating plug is negatively correlated with a thickness of the insulating layer group.

In some embodiments, wherein an insulating connection layer is provided between the first insulating layer and the second insulating layer; and the insulating connection layer includes a first surface and a second surface which are oppositely arranged, the first surface is connected to the first insulating layer, and the second surface is connected to the second insulating layer.

In some embodiments, wherein a thickness of the insulating connection layer is in a range of 1 mm to 2 mm.

In some embodiments, wherein a volume resistivity of the insulating connection layer is equal to or approximately equal to the volume resistivity of the first insulating layer or the second insulating layer.

In some embodiments, wherein lengths of the first insulating layer and the second insulating layer are configured to be positively correlated with a maximum voltage carried by the first wire.

In some embodiments, wherein lengths of the first insulating layer and the second insulating layer are the same.

Embodiments of the present disclosure provide a high-voltage cable, wherein at least one end of the high-voltage cable is equipped with an insulating plug; the insulating plug includes a first wire and an insulating layer group covering the first wire, the insulating layer group including at least two insulating layers; wherein the insulating layer group includes a first insulating layer covering the first wire, and a second insulating layer covering the first insulating layer; and a volume resistivity of the second insulating layer is greater than a volume resistivity of the first insulating layer.

Embodiments of the present disclosure provide an X -ray tube, comprising: a socket device configured to connect an insulating plug, the insulating plug including a first wire and an insulating layer group covering the first wire, the insulating layer group including at least two insulating layers; wherein the insulating layer group includes a first insulating layer covering the first wire, and a second insulating layer covering the first insulating layer; and a volume resistivity of the second insulating layer is greater than a volume resistivity of the first insulating layer; and a second wire disposed in the socket device, wherein the second wire is configured to electrically connect the first wire in the insulating plug after the insulating plug is connected to the socket device, so as to power the X-ray tube.

In some embodiments, wherein the socket device is electrically connected to at least one of a cathode or an anode of the X-ray tube.

In some embodiments, the X-ray tube further comprising a shell configured to accommodate the cathode and the anode of the X-ray tube, wherein the socket device includes a sealing member in sealing connection with the shell, and the sealing member includes a cavity for accommodating the insulating plug.

### BRIEF DESCRIPTION OF THE DRAWINGS

The present disclosure will be further illustrated by way of example embodiments, which will be described in detail with the accompanying drawings. These embodiments are non-limiting. In these embodiments, the same count indicates the same structure, wherein:

FIG. 1 is a schematic diagram illustrating an equivalent circuit of an exemplary insulating layer group in a DC electric field state;

FIG. 2 is a schematic diagram illustrating an application scenario of an exemplary X-ray device according to some embodiments of the present disclosure;

FIG. 3 is a cross-sectional view illustrating an exemplary insulating plug according to some embodiments of the present disclosure;

FIG. 4 is a cross-sectional view illustrating an exemplary insulating connection layer according to some embodiments of the present disclosure;

FIG. 5 is a cross-sectional view illustrating an exemplary insulating plug including an outer insulating layer according to some embodiments of the present disclosure;

FIG. 6 is a cross-sectional view illustrating an exemplary insulating plug including two outer insulating layers according to some embodiments of the present disclosure;

FIG. 7 is a schematic diagram illustrating an electric field distribution of an exemplary insulating plug according to some embodiments of the present disclosure;

FIG. 8 is a side view illustrating an exemplary X-ray tube according to some embodiments of the present disclosure;

FIG. 9 is a side view illustrating an exemplary socket device according to some embodiments of the present disclosure; and

FIG. 10 is a side view illustrating an exemplary socket device according to some embodiments of the present disclosure.

### DETAILED DESCRIPTION

In order to more clearly illustrate the technical solutions of the embodiments of the present disclosure, the following briefly introduces the drawings that need to be used in the description of the embodiments. Apparently, the accompanying drawings in the following description are only some examples or embodiments of the present disclosure, and those skilled in the art can also apply the present disclosure to other similar scenarios according to the drawings without creative efforts. Unless obviously obtained from the context or the context illustrates otherwise, the same numeral in the drawings refers to the same structure or operation.

It should be understood that “system”, “device”, “unit” and/or “module” as used herein is a method for distinguishing different components, elements, parts, portions or assemblies of different levels. However, the words may be replaced by other expressions if other words can achieve the same purpose.

Generally, the words “modules,” “units,” or “blocks” as used herein refer to logic embodied in hardware or firmware, or to a collection of software instructions. The modules, units or blocks described herein may be implemented as software and/or hardware and may be stored in any type of non-transitory computer-readable medium or another storage device. In some embodiments, software modules/units/blocks may be compiled and linked into an executable program. It should be understood that software modules may be called from other modules/units/blocks or from themselves, and/or may be called in response to detected events or interrupts. Software modules/units/blocks configured to execute on a computing device may be provided on a computer-readable medium (e.g., an optical disk, a digital video disk, a flash drive, a magnetic disk, or any other tangible medium), or as a digital download (which may initially be stored in a compressed or installable format that requires installation, decompression, or decryption before execution). The software code here may be partially or completely stored in the storage device of the computing device that performs the operation, and used in the operation of the computing device. Software instructions can be embedded in firmware, such as EPROM. It should also be understood that hardware modules/units/blocks may be included in connected logic components, such as gates and flip-flops, and/or may include programmable units, such as programmable gate arrays or processors. The modules/units/blocks or computing device functions described here may be implemented as software modules/units/blocks, but may be represented in hardware or firmware. Generally, modules/units/blocks described herein refer to logical modules/units/blocks, which may be combined with other modules/units/blocks or divided into sub-modules/sub-units/sub-blocks, notwithstanding that they are physical organizations or storage devices. This description may apply to the system, engine, or part thereof.

It should be understood that when a unit, engine, module, or block is referred to as being “on”, “connected” or “coupled to” another unit, engine, module, or block, unless the context clearly dictates otherwise, the unit, engine, module or block may be directly on, connected to, coupled to, or in communication with another unit, engine, module or block, or there may be intermediate units, engines, modules, or blocks. In the present disclosure, the term “and/or” may include any one or more of the associated listed items or combinations thereof. In the present disclosure, the term “image” refers to a 2D image, a 3D image or a 4D image.

These and other features and characteristics of the present disclosure, the function and operation of the associated structural elements, and the component combinations and manufacturing economies may become more apparent from the following description taken in conjunction with the accompanying drawings, which form a part of the present disclosure. However, it should be understood that the drawings are for the purpose of illustration and description only and are not intended to limit the scope of the disclosure. It should be understood that the drawings are not drawn to scale.

As indicated in the disclosure and claims, the terms “a”, “an”, and/or “the” are not specific to the singular form and may include the plural form unless the context clearly indicates an exception. Generally speaking, the terms “comprising” and “including” only suggest the inclusion of clearly identified steps and elements, and these steps and elements do not constitute an exclusive list, and the method or device may also contain other steps or elements.

The flowchart is used in the present disclosure to illustrate the operations performed by the system according to the embodiments of the present disclosure. It should be understood that the preceding or following operations are not necessarily performed in the exact order. Instead, various steps may be processed in reverse order or simultaneously. Meanwhile, other operations may be added to these procedures, or a certain step or steps may be removed from these procedures.

A metal shell of an X-ray tube may be equipped with an insulating layer group to isolate the metal shell from a high-voltage wire. A distribution of an electric field intensity is extremely uneven along a radial path from the insulating layer group of the X-ray tube to the shell. A region close to the high-voltage wire may be a high field intensity region, and a region far away from the high-voltage wire may be a low field intensity region. Therefore, under the action of a high-voltage electric field, the field intensity of the region close to the high-voltage wire may be very large, causing an insulating material of the insulating layer group to easily age. When the insulating layer group ages and is insufficient to withstand the external high-voltage electric field, insulation breakdown may occur, resulting in damage to the X-ray tube.

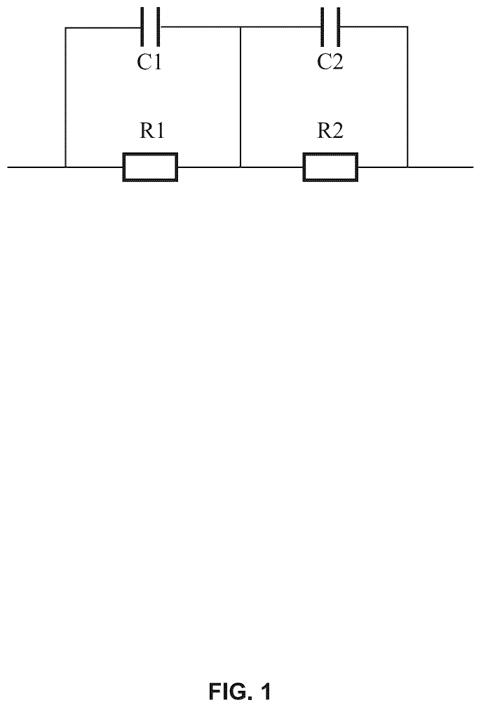

In the prior art, the electric field distribution between the high-voltage wire and the insulating layer group may be uniform through a structural form of capacitive coupling, thereby preventing dielectric breakdown of the insulating layer group. However, the capacitive coupling scheme may be only suitable for an alternating current (AC) electric field. FIG. 1 is a schematic diagram illustrating an equivalent circuit of an insulating layer group in a direct current (DC) electric field state. As shown in FIG. 1, when a DC voltage is applied to both ends of a set of series connected insulating media, a voltage distribution between the insulating media at the moment of voltage application may be inversely proportional to capacitance values of thee insulating media. That is, a voltage at R1 is 2U₀, and a voltage at R2 is U₀. However, charges are polarized under a DC field, and the voltage is gradually become proportional to a resistance value. At this time, the voltage at R1 becomes U₀, and the voltage at R2 becomes 2U₀. Therefore, when the X-ray tube discharges for a long time to form a steady-state DC, the capacitive coupling program may not be able to achieve a uniform electric field.

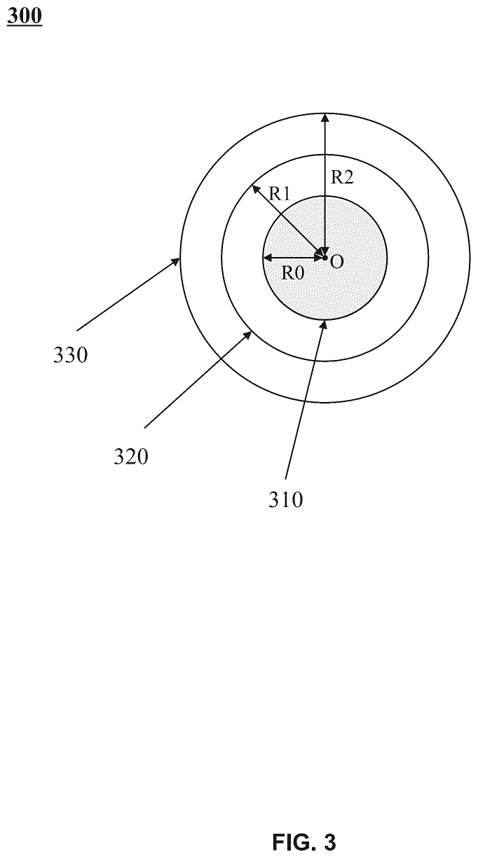

The embodiments of the present disclosure provide an insulating plug comprising a first wire, a first insulating layer covering the first wire, and a second insulating layer covering the first insulating layer. A volume resistivity of the second insulating layer may be greater than a volume resistivity of the first insulating layer. Applying the insulating plug to the connection between a high-voltage generator and the X -ray tube can reduce an average field intensity between the high-voltage wire and the X-ray tube, thereby extending the service life of the insulating material. Moreover, this solution is suitable for the DC electric field and is not limited by the discharge time of the X-ray tube. More descriptions may be found in FIGS. 3-10.

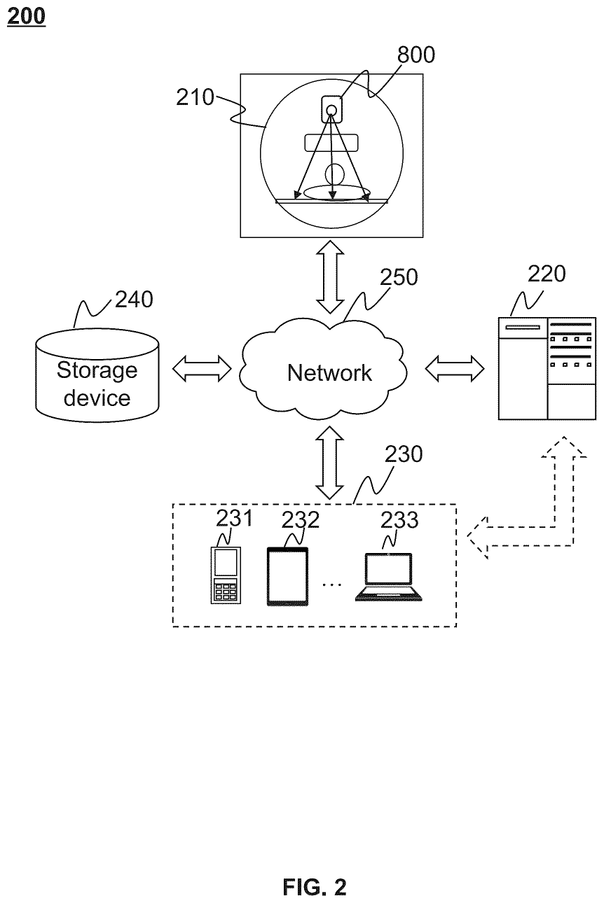

FIG. 2 is a schematic diagram illustrating an application scenario of an exemplary X-ray device according to some embodiments of the present disclosure.

As shown in FIG. 2, in some embodiments, an X-ray system 200 may include an X-ray device 210, a processing device 220, one or more terminal devices 230, a storage device 240, and a network 250. In some embodiments, the X-ray device 210, the processing device 220, the one or more terminal devices 230, and/or the storage device 240 may be connected and/or communicate with each other via a wireless connection, a wired connection, or a combination thereof. In some embodiments, connections in the X-ray system 200 may change. Merely by way of example, as shown in FIG. 2, the X-ray device 210 may be connected to the processing device 220 through the network 250. As another example, the X-ray device 210 may be directly connected to the processing device 220. As another example, the storage device 240 may be connected to the processing device 220 through the network 250 or directly connected to the processing device 220. As another example, the terminal device 230 may be connected to the processing device 220 through the network 250 or directly connected to the processing device 220 (as shown by dotted double-way arrows in FIG. 2), or connected to the X-ray device 210 directly or through the network 250. As another example, the terminal device 230 may be omitted. 230 may be omitted.

In some embodiments, the X-ray device 210 may be an X-ray imaging device. For example, the X-ray imaging device may include a Computed Tomography (CT) device, a Positron Emission Tomography (PET/CT) device, a DSA (digital subtraction angiography) device, a digital X-ray radiography (DR) device, a computed X-ray radiography (CR) device, a digital fluorescence radiography (DF) device, computed tomography equipment, magnetic resonance scanning equipment, a mammography machine, a C-arm scanning device, etc. The X-ray imaging device may be configured to scan an object or a portion thereof located within its detection region and generate an image related to the object or the portion thereof.

In some embodiments, the object may be a biotic or abiotic object. For example, the object may include a patient, an artificial object, or the like. As another example, the object may include a specific part, organ, and/or tissue of the patient. For example, the object may include a head, a neck, a chest, a heart, a stomach, blood vessels, a soft tissue, a tumor, a nodule, or the like, or any combination thereof. In some embodiments, the object may include a region of interest (ROI), e.g., the tumor, the nodule, etc.

In some embodiments, the X-ray device 210 may be an X-ray radiotherapy device. The X-ray radiotherapy device may perform radiotherapy on the object. In some embodiments, the X-ray radiotherapy device may include a single-modal device, e.g., an X-ray therapy device, a Co-60 teletherapy device, a medical electron accelerator, or the like. In some embodiments, the X-ray radiotherapy device may be a multi-modal (e.g., bi-modal) device to obtain a medical image related to the object and perform radiotherapy on the object.

In some embodiments, the X-ray device 210 may be an industrial X-ray device. For example, the X-ray device 210 may be an X-ray detector used for foreign object detection, non-destructive detection, or flaw detection, etc.

In some embodiments, the X-ray device 210 may include an X-ray generation system. The X-ray generation system may include a high voltage generator (HVG), a high-voltage cable, and an X-ray tube (e.g., an X-ray tube 800).

The high-voltage cable may be configured to connect the high voltage generator and the X-ray tube. The high voltage generator may generate a high voltage or strong current and transmit the high voltage or the strong current to the X-ray tube through the high-voltage cable. The high voltage or the strong current may be transmitted to an emission cathode of the X-ray tube to apply a negative high voltage to the emission cathode of the X-ray tube, and may be transmitted to an anode target disk of the X-ray tube to apply a positive high voltage to the anode target disk of the X-ray tube. Accordingly, electrons emitted from the emission cathode may bombard the anode target disk under the action of the electric field to generate X-rays.

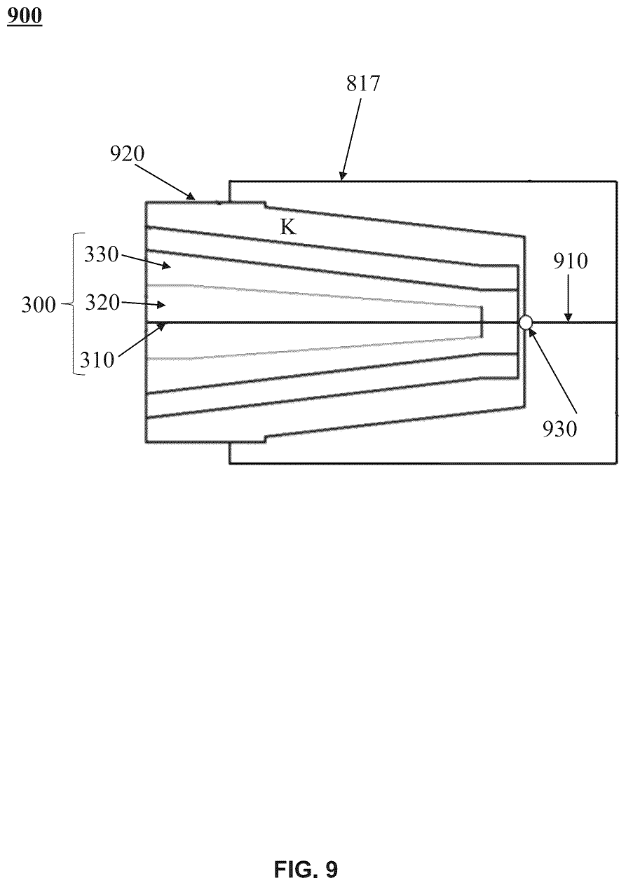

In some embodiments, the high voltage generator may include at least one socket device (e.g., a socket device 900) configured to connect an insulating plug (e.g., an insulating plug 300 at one end of the high-voltage cable). The insulating plug may include a first wire 310, a first insulating layer 320 covering the first wire 310, and a second insulating layer 330 covering the first insulating layer 320. A volume resistivity of the second insulating layer may be greater than a volume resistivity of the first insulating layer. More descriptions regarding the insulating plug may be found in the relevant descriptions of FIG. 3. For example, one end of the high voltage generator connected to the high-voltage cable may be equipped with a socket device 900. The socket device 900 may be internally provided with a second wire 910. When the socket device 900 is connected to the insulating plug 300 on the high-voltage cable, the second wire 910 may be configured to be electrically connected to the first wire 310. More descriptions may be found in descriptions of FIG. 9.

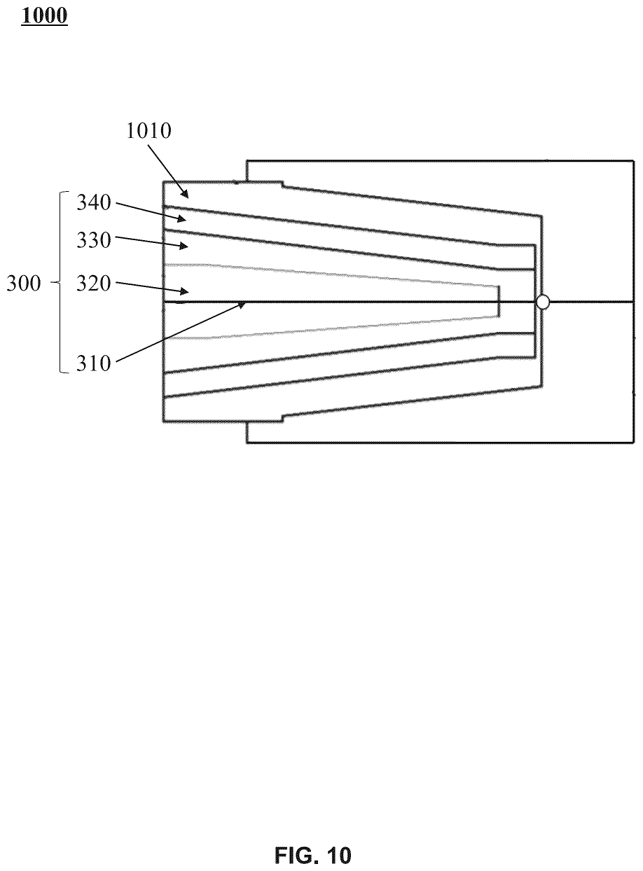

In some embodiments, the high voltage generator may include a socket device which is provided with the insulating plug 300. For example, one end of the high voltage generator connected to the high-voltage cable may be provided with a socket device 1000 which is internally provided with the insulating plug 300. More descriptions may be found in descriptions of FIG. 10.

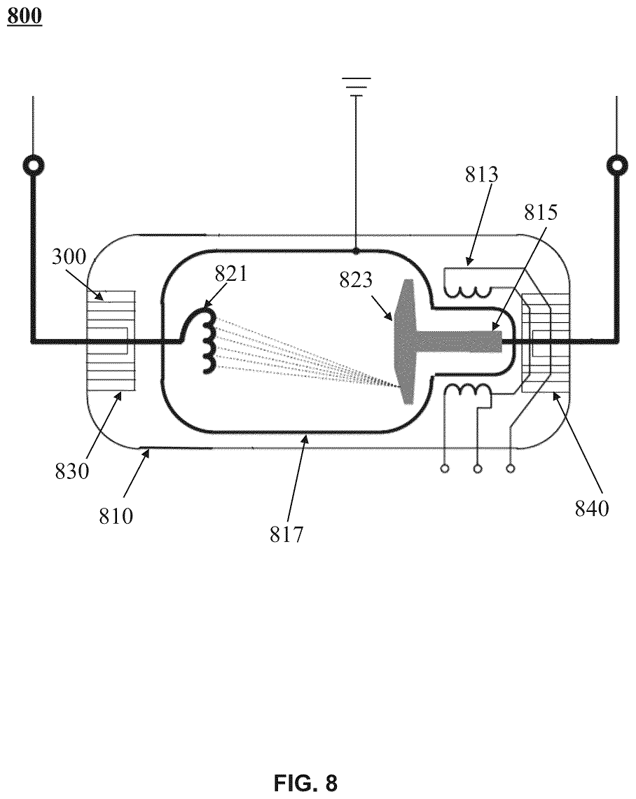

In some embodiments, the X-ray tube may include at least one socket device 1000 which is provided with the insulating plug. In some embodiments, the X-ray tube may include at least one socket device 900 configured to connect the insulating plug. More descriptions regarding the X-ray tube may be found in FIG. 8.

In some embodiments, at least one end of the high-voltage cable may be provided with an insulating plug (e.g., the insulating plug 300). For example, when both the high voltage generator and the X-ray tube include the socket device 900, both ends of the high-voltage cable may be provided with the insulating plug 300 for connecting the high voltage generator and the X-ray tube. As another example, when one of the high voltage generators and the X-ray tube includes the socket device 900, one end of the high-voltage cable may be provided with the insulating plug 300. One end of the high-voltage cable may be configured to connect the socket device 1000.

In some embodiments, the high-voltage cable may not include an insulating plug. For example, when both the high voltage generator and the X-ray tube include the socket device 1000, the high-voltage cable configured to connect the X-ray tube and the high voltage generator may not include the insulating plug 300.

The processing device 220 may process data and/or information obtained from the X-ray device 210, the storage device 240, and/or the terminal device(s) 230. For example, the processing device 220 may generate a reconstructed image of the object based on projection data of the object scanned by the X-ray device 210. In some embodiments, the processing device 220 may be integrated into X-ray device 210.

The terminal device 230 may be connected to and/or communicate with the X-ray device 210, the processing device 220, and/or the storage device 240. For example, the terminal device 230 may obtain a processed image from the processing device 220. As another example, the terminal device 230 may obtain the projection data through the X-ray device 210 and transmit the projection data to the processing device 220 for processing. In some embodiments, the terminal device 230 may include a mobile device 231, a tablet computer 232, a laptop computer 233, or the like, or any combination thereof.

The storage device 240 may store data and/or instructions. In some embodiments, the storage device 240 may store data obtained from the X-ray device 210, the processing device 220, and/or the terminal device 230. In some embodiments, the storage device 240 may store data and/or instructions that processing device 220 may perform or be used to perform exemplary embodiments described in the present disclosure. In some embodiments, the storage device 240 may include a mass storage device, a removable storage device, a volatile read-write storage device, a read-only memory device (ROM), or the like, or any combination thereof. In some embodiments, the storage device 240 may be implemented on a cloud platform.

In some embodiments, the storage device 240 may be connected to the network 250 to communicate with at least one component (e.g., the processing device 220, and the terminal device 230) of the X-ray system 200. The at least one component of the X-ray system 200 may access the data or the instructions stored in the storage device 240 through the network 250.

The network 250 may include any suitable network that facilitates information and/or data exchange of the X-ray system 200. In some embodiments, the at least one component (e.g., the X-ray device 210, the processing device 220, the terminal device 230, the storage device 240, etc.) of the X-ray system 200 may transmit information and/or data to the at least one other component of the X-ray system 200 through the network 250. For example, the processing device 220 may obtain the projection data from the X-ray device 210 through the network 250. As another example, the processing device 220 may obtain user instructions from the terminal device 230 through the network 250. In some embodiments, the network 250 may be any type of wired or wireless network, or a combination thereof. In some embodiments, the network 250 may include at least one network access point. For example, the network 250 may include at least one wired or wireless network access point, e.g., a base station and/or an Internet exchange point, through which the at least one component of the X-ray system 200 may be connected to network 250 for data and/or or information exchange.

It should be noted that the above description is provided for illustrative purposes only and is not intended to limit the scope of the present disclosure. For those having ordinary skills in the art, various changes and modifications can be made under the guidance of the contents of the present disclosure. The features, structures, methods, and other characteristics of the exemplary embodiments described in the present disclosure may be combined in various ways to obtain additional and/or alternative exemplary embodiments. For example, the X-ray system 200 may further include a display device for outputting and displaying the reconstructed image generated by the processing device 220. However, such changes and modifications do not depart from the scope of the present disclosure.

FIG. 3 is a cross-sectional view illustrating an exemplary insulating plug according to some embodiments of the present disclosure.

In some embodiments, the insulating plug 300 may include a first wire, and an insulating layer group covering the first wire. The insulating layer group may include at least two insulating layers. As shown in FIG. 3, the insulating plug 300 may include a first wire 310, a first insulating layer 320 covering the first wire 310, and a second insulating layer 330 covering the first insulating layer 320.

In some embodiments, volume resistivities of the at least two insulating layers may increase layer by layer along a radius direction (hereinafter referred to as a radial direction) of the first wire. As shown in FIG. 3, the volume resistivity of the second insulating layer 330 may be greater than the volume resistivity of the first insulating layer 320. In some embodiments, the volume resistivity may be increased in a manner including, but not limited to, a gradient increase and/or a non-gradient increase.

Based on the above, on a radial path of a high-voltage wire, a region close to the high-voltage wire may be a high field intensity region, and a region far away from the high-voltage wire may be a low field intensity region, i.e., a distribution of electric field intensities on this path is non-uniform. When the insulating plug 300 is applied to the X-ray device, one end of the first wire 310 may be connected to the high-voltage wire, so the distribution of the electric field intensities on the path from the first wire 310 to the first insulating layer 320 and then to the second insulating layer 330 may be non-uniform. By arranging an insulating material with a relatively small volume resistivity in an insulating layer (e.g., the first insulating layer 320) close to the first wire and arranging an insulating material with a relatively large volume resistivity in an insulating layer (e.g., the second insulating layer 330), far away from the first wire, the electric field intensity of the high field intensity region may be decreased, and the electric field intensity of the low field intensity region may be increased, thereby making the field intensity distribution on the radial path of the high-voltage wire more uniform.

In some embodiments, a radius of each of the at least two insulating layers (e.g., the first insulating layer 320 and the second insulating layer 330) of the insulating plug may be a distance between an outer surface of the insulating layer and a center line of the first wire along a length direction, wherein the insulating layer is directly connected to the first wire and the at least two insulating layers are directly connected to each other. The length direction of the first wire refers to a direction formed by two ends (e.g., two ends of a wire connecting the X-ray tube and the high voltage generator) of the wire used to achieve an electrical connection. The outer surface of the insulating layer refers to a surface of the insulating layer away from the first wire. For example, as shown in FIG. 3, the first insulating layer 320 is directly connected to the first wire 310, and the second insulating layer 330 is directly connected to the first insulating layer 320, a radius R1 of the first insulating layer 320 may be a vertical distance from the point O on the center line of the first wire 310 along the length direction to the outer surface of the first insulating layer 320, and a radius R2 of the second insulating layer 330 may be a vertical distance from the point O on the center line of the first wire 310 to the outer surface of the second insulating layer 330.

In some embodiments, the field intensities at the outer surfaces of the at least two insulating layers may be equal or approximately equal. For example, as shown in FIG. 3, the field intensity at the outer surface of the first insulating layer 320 and the field intensity at the outer surface of the second insulating layer 330 may be equal or approximately equal.

In some embodiments, a ratio of the volume resistivity of the first insulating layer 320 to the volume resistivity of the second insulating layer 330 may be a ratio of the radius of the first wire 310 to the radius of the first insulating layer 320, i.e., ρ1:ρ2=R0:R1. ρ1 represents the volume resistivity of the first insulating layer 320, ρ2 represents the volume resistivity of the second insulating layer 330, R0 represents the radius of the first wire 310, and R1 represents the radius of the first insulating layer 320.

By configuring the ratio of the volume resistivities of the plurality of insulating layers as the ratio of the radii of the plurality of insulating layers, the distribution of the electric field intensities between the first wire and the plurality of insulating layers may be better averaged, so that the field intensity distribution along the radial path of the high-voltage wire may be more uniform.

The greater the voltage carried on the high-voltage wire, the longer a safety distance (i.e., a cable length) of the high-voltage cable is required. In order to ensure the safety of the wire, in some embodiments, lengths of the at least two insulating layers of the insulating plug may be configured to be positively correlated with a maximum voltage carried by the first wire. Specifically, the greater the maximum voltage carried by the first wire, the longer the lengths of the at least two insulating layers covering the first wire. When the insulating plug is applied to an X-ray scenario, after a voltage is applied to the X-ray tube, the voltage carried by the first wire may also change as time changes. The maximum voltage refers to a maximum voltage carried by the first wire as time changes after the voltage is applied to the X-ray tube. The length of the insulation layer refers to a distance along the length direction of the first wire.

Referring to FIG. 3, the lengths of the first insulating layer 320 and the second insulating layer 330 may be configured to be positively correlated with the maximum voltage carried by the first wire 310. For example, the maximum voltage carried by the first wire 310 may be determined according to a working scenario (e.g., an X-ray imaging device, an X-ray radiotherapy device, or an X-ray industrial device) of the insulating plug 300, and then the lengths of first insulating layer 320 and the second insulating layer 320 may be determined.

In some embodiments, the lengths of the at least two insulating layers of the insulating plug may be the same. Specifically, the lengths of the first insulating layer 320 and the second insulating layer 330 may be the same. The lengths of the first insulating layer 320 and the second insulating layer 330 may be the same, so that the distribution of the electric field intensities around the first wire 310 may be more uniform, and non-uniform electric field distribution may be effectively avoided.

In some embodiments, the lengths of the at least two insulating layers of the insulating plug may be different. Specifically, the lengths of the at least two insulating layers may increase or decrease in sequence along the radial direction of the first wire. For example, the length of the first insulating layer 320 may be greater than the length of the second insulating layer 330, or the length of the first insulating layer 320 may be less than the length of the second insulating layer 330.

In some embodiments, lengths of at least two insulating layer groups of the at least two insulating layers may be different. Specifically, when the insulating layer group includes three or more insulating layers, the three or more insulating layers may be divided into the at least two insulating layer groups, and the lengths of the at least two insulating layer groups may be different. The insulating layers included in each of the at least two insulating layer groups may be connected in sequence in a covering manner. For example, the three or more insulating layers may be divided into a first insulating layer group and a second insulating layer group, lengths of all the insulating layers included in the first insulating layer group may be the same (e.g., a first length), lengths of all the insulating layers included in the second insulating layer group may be the same (e.g., a second length), and the first length and the second length may be different values. In some embodiments, the lengths of the at least two insulating layer groups may increase or decrease in sequence along the radial direction.

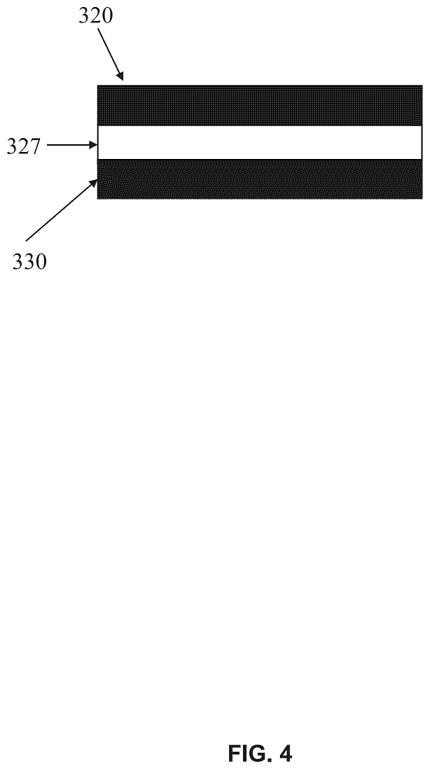

In some embodiments, an insulating connection layer (also referred to as a first insulating connection layer) may be provided between the first insulating layer 320 and the second insulating layer 330. The insulating connection layer may include a first surface and a second surface which are oppositely arranged. The first surface may be connected to the first insulating layer 320, and the second surface may be connected to the second insulating layer 330. As shown in FIG. 4, an insulating connection layer 327 may be provided between the first insulating layer 320 and the second insulating layer 330.

In some embodiments, a thickness of the insulating connection layer may be set according to a total thickness of the first insulating layer 320 and the second insulating layer 330. For example, the smaller the total thickness of the first insulating layer 320 and the second insulating layer 330, the smaller the thickness of the insulating connection layer. In some embodiments, the thickness of the insulating connection layer may be in a range of 1 mm to 2 mm. The thickness of the insulating connection layer refers to a vertical distance between the first surface of the insulating layer connected to the first insulating layer and the second surface of the insulating layer connected to the second insulating layer. By setting the thickness of the insulating connection layer to be in the range of 1 mm to 2 mm, it is possible to avoid affecting the insulation performance or uniform field intensity performance of the first insulating layer and the second insulating layer due to an excessive thickness of the insulating connection layer.

In some embodiments, a volume resistivity of the insulating connection layer may be equal to or approximately equal to the volume resistivity of the first insulating layer 320. In some embodiments, the volume resistivity of the insulating connection layer may be equal to or approximately equal to the volume resistivity of the second insulating layer 330. For example, the volume resistivity of the insulating connection layer may be equal to the volume resistivity of the first insulating layer 320, or slightly greater/slightly less than the volume resistivity of the first insulating layer 320. By setting the volume resistivity of the insulating connection layer to be the equal to or approximately equal to the volume resistivity of the first insulating layer or the volume resistivity of the second insulating layer, it is possible to avoid a sudden change in an electric field between the first insulating layer and the second insulating layer due to a sudden change in the volume resistivity at the insulating connection layer.

In some embodiments, an insulating material of the insulating connection layer may be determined based on the volume resistivity. For example, if it is determined that the volume resistivity of the insulating connecting layer is equal to or approximately equal to the volume resistivity of the first insulating layer, the insulating connecting layer and the first insulating layer may use a same insulating material, or an insulating material with an equal or approximately equal volume resistivity.

Serving as a buffer layer between the first insulating layer 320 and the second insulating layer 330, the insulating connection layer enables a better connection between the first insulating layer 320 and the second insulating layer 330. In addition, the material of the insulating connection layer is the insulating material, and the volume resistivity is set to be equal to or approximately equal to that of the connected first insulating layer or the second insulating layer. The connection between the first insulating layer 320 and the second insulating layer 320 is enabled to be stronger using the principle of principle of similarity and solubility, thereby improving the insulation performance of the insulating plug 300.

In some embodiments, the first wire 310 of the insulating plug 300 may be provided with a terminal used for an electrical connection with a second wire of a socket device (e.g., the socket device 900).

It should be noted that the above description of the insulating plug 300 is for illustrative purposes only and is not intended to limit the scope of the present disclosure. For those having ordinary skills in the art, various variations and modifications can be made based on the present disclosure. For example, the insulating plug 300 may include a first insulating layer, a second insulating layer, a third insulating layer, etc. An insulating connection layer may be provided between the second insulating layer and the third insulating layer. For example, there may be no insulating connecting layer between the first insulating layer and the second insulating layer. However, these changes and modifications do not depart from the scope of the present disclosure.

In some embodiments, the insulating layer group of the insulating plug 300 may include at least one outer insulating layer covering the second insulating layer 330. In some embodiments, a volume resistivity of the at least one outer insulating layer may be greater than the volume resistivity of the second insulating layer 330.

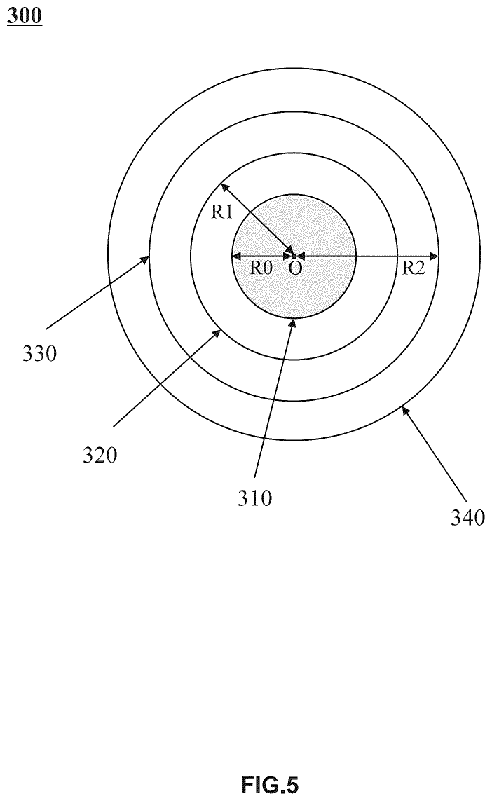

FIG. 5 is a cross-sectional view illustrating an exemplary insulating plug including an outer insulating layer according to some embodiments of the present disclosure. As shown in FIG. 5, in some embodiments, the insulating plug 300 may include an outer insulating layer 340 covering the second insulating layer 330. A volume resistivity of the outer insulating layer 340 may be greater than the volume resistivity of the second insulating layer 330.

In some embodiments, a ratio of the volume resistivity of the second insulating layer to the volume resistivity of one of the at least one outer insulating layer closest to the second insulating layer may be a ratio of the radius of the first insulating layer to the radius of the second insulating layer. Referring to FIG. 5, the outer insulating layer 340 may cover the second insulating layer 330 and may be closest to the second insulating layer 330. The ratio of the volume resistivity of the outer insulating layer 340 to the volume resistivity of the second insulating layer 330 may be a ratio of the radius of the second insulating layer 330 to the radius of the first insulating layer 320.

In some embodiments, a ratio of the volume resistivity of the first insulating layer to the volume resistivity of one of the at least one outer insulating layer (e.g., the outer insulating layer 340) closest to the second insulating layer 330 may be a ratio of the radius of the first wire to the radius of the second insulating layer.

In some embodiments, a ratio of the volume resistivity of the first insulating layer 320 to the volume resistivity of the second insulating layer 330 and the volume resistivity of one of the at least one outer insulating layer (e.g., the outer insulating layer 340) closest to the second insulating layer 330 may be a ratio of the radius of the first wire to the radius of the first insulating layer and the radius of the second insulating layer. For example, as shown in FIG. 5, the ratio of the volume resistivity of the first insulating layer 320 to the volume resistivity of the second insulating layer 330 and the volume resistivity of the outer insulating layer 340 closest to the second insulating layer may be ρ1:ρ2:ρ3=R0:R1: R2. R2 is the radius of the second insulating layer 330, and ρ3 is the volume resistivity of the outer insulating layer 340.

In some embodiments, an insulating connection layer (also referred to as a second insulating connection layer) may also be provided between the second insulating layer 330 and the outer insulating layer (e.g., the outer insulating layer 340) of the at least one outer insulating layer closest to the second insulating layer. The second insulating connection layer may include a first surface and a second surface which are oppositely arranged. The first surface may be connected to the outer insulating layer 340, and the second surface may be connected to the second insulating layer 330. In some embodiments, a thickness of the second insulating connection layer may be in a range of 1 mm to 2 mm. In some embodiments, a volume resistivity of the second insulating connection layer may be equal to or approximately equal to the volume resistivity of the outer insulating layer 340 or the volume resistivity of the second insulating layer 330.

In order to better carry the voltage of the first wire 310, and improve the insulation effect and uniform electric field capability of the insulating layer group to avoid breakdown of the insulating layer, in some embodiments, the insulating layer group of the insulating plug 300 may include at least two outer insulating layers covering the second insulating layer 330. In some embodiments, a volume resistivity of a second outer insulating layer of the at least two outer insulating layers may be greater than a volume resistivity of a first outer insulating layer of the at least two outer insulating layers. The first outer insulating layer may cover the second insulating layer 330 and the second outer insulating layer may cover the first outer insulating layer.

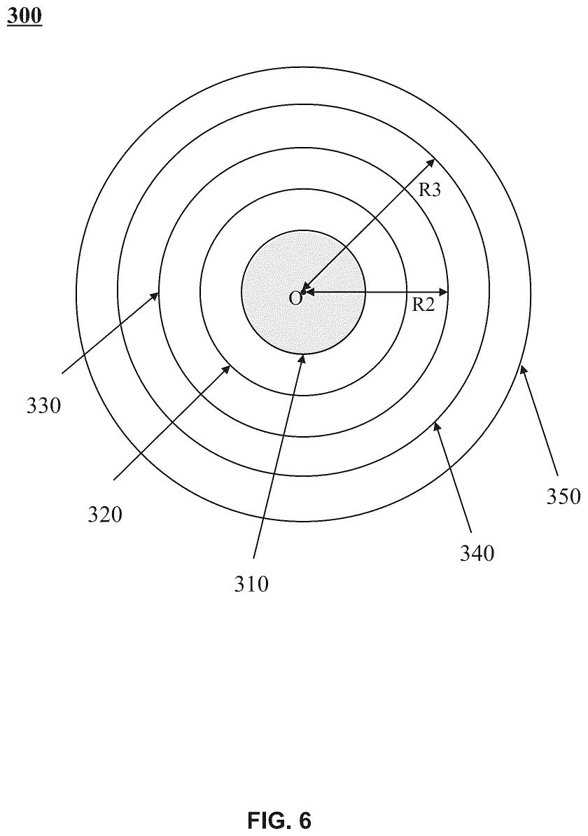

FIG. 6 is a cross-sectional view illustrating an insulating plug including two outer insulating layers. As shown in FIG. 6, in some embodiments, the insulating plug 300 may include an outer insulating layer 340 and an outer insulating layer 350 covering the second insulating layer 330. The outer insulating layer 350 (i.e., the second outer insulating layer) may cover the outer insulating layer 340 (i.e., the first outer insulating layer). A volume resistivity of the outer insulating layer 350 may be greater than a volume resistivity of the outer insulating layer 340.

In some embodiments, a ratio of the volume resistivity of the first outer insulating layer to the volume resistivity of the second outer insulating layer may be a ratio of a radius of the second insulating layer to a radius of the first outer insulating layer. For example, as shown in FIG. 6, the ratio of the volume resistivity of the outer insulating layer 340 to the volume resistivity of the outer insulating layer 350 may be ρ3:ρ4=R2:R3. R3 refers to the radius of the outer insulating layer 340, and ρ4 refers to the volume resistivity of the outer insulating layer 350.

In some embodiments, an insulating connection layer (also referred to as a third insulating connection layer) may also be provided between the first outer insulating layer and the second outer insulating layer. The third insulating connection layer may include a first surface and a second surface which are oppositely arranged. The first surface may be connected to the first outer insulating layer (e.g., the outer insulating layer 340), and the second surface may be connected to the second outer insulating layer (e.g., the outer insulating layer 350). In some embodiments, a thickness of the third insulating connection layer may be in a range of 1 mm to 2 mm. In some embodiments, a volume resistivity of the third insulating connection layer may be equal to or approximately equal to the volume resistivity of the first outer insulating layer or the volume resistivity of the second outer insulating layer.

The insulating layer group covering the first wire 310 is configured as a composite structure including the plurality of insulating layers (e.g., the first insulating layer 320, the second insulating layer 330, the outer insulating layer 340, and the outer insulating layer 350), and the volume resistivity of each insulating layer increases along the radial direction based on the radius, so that the electric field intensity of the high field intensity region can be reduced, and the electric field intensity of the low field intensity region can be increased, thereby making the overall distribution of the electric field intensity uniform, and extending the service life of the insulating plug.

In some embodiments, a thickness of the insulating layer group (e.g., the first insulating layer 320, the second insulating layer 330, the outer insulating layer 340, and the outer insulating layer 350) of the insulating plug 300 may be configured to be positively correlated with a maximum voltage carried by the first wire 310. The greater the maximum voltage carried by the first wire 310, the greater the thickness of the insulating layer group. The thickness of the insulating layer group refers to the radial distance along the insulating layer group.

In some embodiments, at a certain voltage, an average value of the electric field intensity at the insulating plug may be negatively correlated with the thickness of the insulating layer group. The average value of the electric field intensity of the insulating plug refers to an average value of an electric field intensity in a region formed in a length direction and/or a radial direction of the insulating layer group. For example, if a length of each insulating layer (e.g., the first insulating layer, the second insulating layer, the first outer insulating layer, and the second outer insulating layer) of the insulating layer group is 1 m, and the thickness of the insulating layer group (including, for example, the thickness of the first insulating layer, the second insulating layer, the first outer insulating layer, the second outer insulating layer, and the insulating connection layer) is 9 mm, the average value of the electric field intensity of the insulating plug may be an average value of an electric field intensity of a cylindrical region 1 m long and 9 mm in radius. For example, if the thickness of the insulating layer group (including the thickness of the first insulating layer, the second insulating layer, the first outer insulating layer, the second outer insulating layer, and the insulating connection layer) is 9 mm, the average value of the electric field intensity of the insulating plug may be an average value of the electric field intensity of a circular region 9 mm in radius. Therefore, the greater the thickness of the insulating layer group, the smaller the average value of the electric field intensity at the insulating plug.

Based on the above content, in some embodiments, the lengths of the insulating layers of the insulating layer group may be determined based on the maximum voltage carried by the first wire.

In some embodiments, the maximum voltage carried by the first wire may be determined according to an X-ray device to which the insulating plug is applicable. For example, an operating voltage of X-ray imaging device is generally less than 150 kV. Correspondingly, the maximum voltage carried by the first wire of the insulating plug configured in the X-ray imaging device is less than 150 kV. As another example, an operating voltage of an X-ray radiotherapy device is greater than 150 kV. Correspondingly, the maximum voltage carried by the first wire of the insulating plug configured in the X-ray radiotherapy device is greater than 150 kV. As another example, an operating voltage of an X-ray industrial device is 300 kV. Correspondingly, the maximum voltage carried by the first wire of the insulating plug configured in the X-ray industrial device is 300 kV.

In some embodiments, when the lengths of the insulating layers included in the insulating layer group are different, a minimum length of the shortest insulating layer may be determined based on the maximum voltage carried by the first wire. Further, the lengths of other insulating layers may be determined based on the minimum length. For example, a length of the innermost first insulating layer 320 may be determined first, and then lengths of the second insulating layer 330, the outer insulating layer 340 (i.e., the first outer insulating layer) and the outer insulating layer 350 (i.e., the second outer insulating layer) may be determined in an ascending order from the inside to the outside along the radial direction of the insulating plug 300. As another example, the length of the outermost outer insulating layer 350 may be determined first, and then the lengths of the outer insulating layer 340, the second insulating layer 330, and the third insulating layer 340 may be determined in a descending order from the inside to the outside along the radial direction of the insulating plug 300.

In some embodiments, the thickness of the insulating layer group may be determined based on the maximum voltage carried by the first wire. The greater the maximum voltage, the greater the thickness of the insulating layer group. In some embodiments, the thickness of the insulating layer group may be in a preset range, such as in a range of 9-23 mm.

In some embodiments, a thickness (i.e., the radius) and/or a count of layers of a single insulating layer may be determined based on the thickness of the insulating layer group. For example, when the thickness of the single insulating layer is less than or equal to ½ of the thickness of the insulating layer group, the insulating layer group may include two insulating layers (e.g., the first insulating layer 320 and the second insulating layer 330). As another example, when the thickness of the single insulating layer is less than or equal to ⅓ of the thickness of the insulating layer group, the insulating layer group may include three insulating layers (e.g., the first insulating layer 320, the second insulating layer 330, and the outer insulating layer 340).

If the thickness of the single insulating layer is too small, the single insulating layer may be easily broken down, resulting in reduced safety of the insulating layer group. If the thickness of the single insulating layer is too large, uniformity of the electric field of the insulating plug may be affected.

In some embodiments, the volume resistivity of the insulating layer may be determined first, and the thickness of the single insulating layer and the count of layers of the insulating layer group may be determined based on the volume resistivity. For example, if the volume resistivity ρ1 of the first insulating layer 320 is in a range of 0.8*10¹² Ω·m−1.2*10¹² Ω·m, the volume resistivity ρ2 of the second insulating layer 330 is in a range of 1.8*10¹² Ω−2.2*10¹² Ω·m, and the volume resistivity ρ3 of the outer insulating layer 340 is in a range of 2.8*10¹² Ω·m−3.2*10¹² Ω·m, the thickness of each insulating layer may be determined according to the formula ρ1:ρ2:ρ3=R1:R2:R3.

In some embodiments, the volume resistivity of the first insulating layer may be inversely proportional to the maximum voltage carried by the first wire. The greater the maximum voltage carried by the first wire 310, the smaller the volume resistivity of the insulating material may be for the first insulating layer 320 covering the first wire 310. In this way, the electric field intensity at the first insulating layer may be further reduced, i.e., the electric field intensity of the high field intensity region may be further reduced, thereby reducing the average value of the electric field intensity at the insulating plug. The volume resistivity of the insulating material may be obtained by looking up a table. After the volume resistivity of the first insulating layer 320 is determined, the volume resistivities of the second insulating layer 330, the outer insulating layer 340, and the outer insulating layer may be further determined in such a manner that the volume resistivity of the insulating layer increases layer by layer along the radial direction of the first wire 310.

In some embodiments, the thickness of the first insulating layer 320 may be determined first based on the maximum voltage carried by the first wire 310. For example, the greater the maximum voltage carried by the first wire, the greater the thickness of the first insulating layer 320 (i.e., the greater the value of the radius R1), so as to better reduce electric field intensity at the first insulating layer 320. Further, the thickness of each insulating layer (e.g., the second insulating layer 330, the outer insulating layer 340, and the outer insulating layer 350) may be determined based on the relationship between the volume resistivity and the radius.

In some embodiments, the radius of each insulating layer may be determined first, and the volume resistivity of each insulating layer may be determined based on the radius. For example, after the thickness and the count of layers of the insulating layer group are determined, the thickness of each insulating layer may be determined layer by layer in an ascending order along the radial direction of the first wire 310. Further, the volume resistivity of each insulating layer may be determined based on the relationship between the volume resistivity and the radius.

In some embodiments, after the total thickness of the insulating layer group, the thickness of the single insulating layer, the count of insulating layers, and the volume resistivity are determined, the performance of the insulating layer group may be verified. For example, a maximum field intensity at the insulating plug may be determined first according to a working environment of the X-ray device to which the insulating plug is applicable, whether the maximum field intensity is smaller than a maximum breakdown field intensity of the insulating layer may be determined, and whether the average value of the electric field intensity of the insulating plug satisfies a preset condition (e.g., whether the average value is less than a preset value) may be determined. If all are satisfied, it is determined that the current insulating layer group satisfies the working requirements. Otherwise, the total thickness of the insulating layer group, the thickness of the single insulating layer, the count of insulating layers, or the volume resistivity may be readjusted until the above conditions are satisfied.

In some embodiments, the plurality of insulating layers (e.g., the first insulating layer 320, the second insulating layer 330, the outer insulating layer 340, and the outer insulating layer 350) of the insulating layer group may use two different insulating materials, or use a same insulation material. In some embodiments, the insulating material may include insulating oil (e.g., natural mineral oil, natural vegetable oil, or synthetic oil), insulating paint, insulating paste, or insulating gas (e.g., sulfur hexafluoride). For example, the insulating material of the insulating layer may be determined based on the volume resistivity. If the first insulating layer 320 and the second insulating layer 330 use the same insulating material, a percentage of a main content of the insulating material may be set to be different to ensure that the volume resistivity of the second insulating layer 330 is greater than the volume resistivity of the first insulating layer 320.

In some embodiments, when the insulating layer group of the insulating plug 300 is prepared, either a layered preparation process or an integrated preparation process may be used. It should be noted that when the integrated preparation process is adopted, the first insulating layer, the second insulating layer, the first outer insulating layer and the second outer insulating layer may be distinguished according to a change in volume resistivity. For example, the change in volume resistivity may occur at an interface between the first insulating layer 320 and the second insulating layer 330, so that the first insulating layer and the second insulating layer may be distinguished.

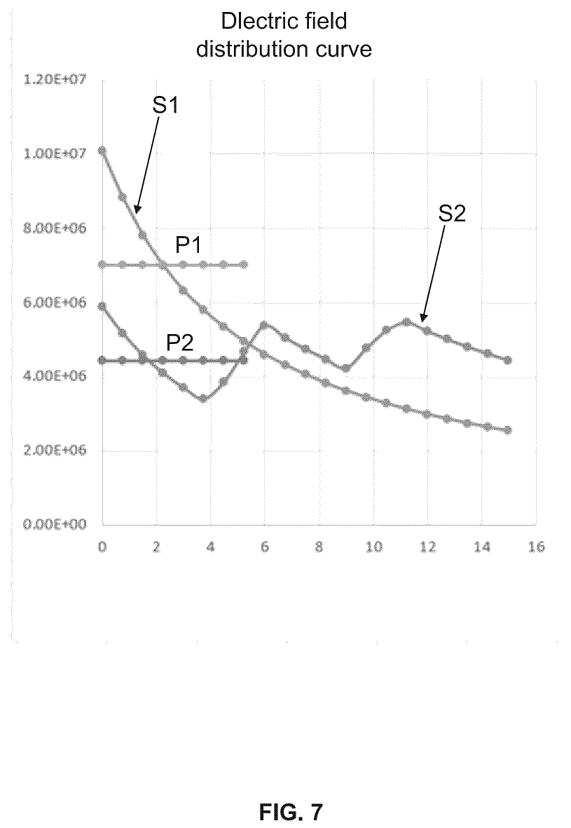

FIG. 7 is a schematic diagram illustrating an electric field distribution of an insulating plug. The vertical axis represents the electric field intensity, and the horizontal axis represents the distance away from the center line of the first wire along the radial direction. S1 represents an electric field distribution curve corresponding to a single insulating layer, and S2 represents an electric field distribution curve corresponding to an insulating plug using an insulating layer group described in the embodiments. P1 represents an average electric field intensity of the curve S1, and P2 represents an average electric field intensity of the curve S2. Referring to FIG. 7, compared with the insulating plug using the single insulating layer, the electric field distribution intensity of the insulating plug using the insulating layer group including the at least two insulating layers may decrease in a larger proportion, and the average electric field intensity may decrease significantly, so that the performance of the uniform electric field distribution may be better.

It should be noted that the above description of the insulating plug 300 is for illustrative purposes only and is not intended to limit the scope of the present disclosure. For those having ordinary skills in the art, various variations and modifications can be made based on the present disclosure. For example, the insulating plug 300 may include at least one outer insulating layer covering a third insulating layer. As another example, the insulating plug 300 may include three outer insulating layers covering the second insulating layer, and the three outer insulating layers may be connected in sequence in a covering manner. However, these changes and modifications do not depart from the scope of the present disclosure.

The embodiments of the present disclosure further provide an X-ray tube including at least one socket device.

FIG. 8 is a side view illustrating an exemplary X-ray tube according to some embodiments of the present disclosure.

As shown in FIG. 8, in some embodiments, an X-ray tube 800 may include a housing 810 configured to accommodate all components of the X-ray tube. Specifically, the housing 810 may include a stator 813, a rotor 815, a cathode 821, an anode 823, a first socket device 830, and a second socket device 840. The rotor 815 may drive a target disk of an anode 823 to rotate.

In some embodiments, the socket device may be electrically connected to at least one of the cathodes or the anode of the X-ray tube. As shown in FIG. 8, the first socket device 830 may be electrically connected to the cathode 821, and the second socket device 840 may be electrically connected to the anode 823. In some embodiments, the X-ray tube 800 may include only one socket device electrically connected to the cathode 821 and the anode 823. That is, one socket device may be simultaneously connected to the cathode and the anode of the X-ray tube. In some embodiments, the X-ray tube 800 may only include the first socket device 830 electrically connected to the cathode 821.

In some embodiments, a structure of the first socket device 830 or the second socket device 840 of the X-ray tube 800 may be a structure of the socket device 900 shown in FIG. 9. In this case, the socket device (e.g., the first socket device 830 and the second socket device 840) of the X-ray tube 800 may be configured to connect the insulating plug 300. The socket device 900 may be provided with a second wire configured to electrically connect the first wire in the insulating plug after the insulating plug 300 is connected to the socket device 900 (e.g., the first socket device 830 and the second socket device 840), so as to power the X-ray tube. More descriptions may be found in the descriptions of FIG. 9.

In some embodiments, the structure of the first socket device 830 or the second socket device 840 of the X-ray tube 800 may be the structure of the socket device 1000 shown in FIG. 10. The socket device 1000 may include a structure similar to the insulating plug 300. In this case, the socket device of the X-ray tube 800 may be directly connected to a high-voltage wire. Another end of the high-voltage wire may be connected to a high voltage generator. By arranging the jacket device connected to the insulating plug 300 in the X-ray tube, the electric field intensity distribution between the X-ray tube and the high-voltage cable is enabled to be uniform, insulation breakdown at the connection of the X-ray tube caused by non-uniform electric field intensity distribution can be avoided, and the stability and effectiveness of the X-ray tube can be improved.

In some embodiments, as shown in FIG. 8, the X-ray tube 800 may further include a shell 817 configured to accommodate the cathode 821 and the anode 823. In some embodiments, the socket device (e.g., the first socket device 830 or the second socket device 840) may include a sealing member in sealing connection with the shell 817. The sealing member may form a cavity for accommodating the insulating plug (e.g., insulating plug 300) of the high-voltage cable.

In some embodiments, as shown in FIG. 8, after the insulating plug 300 including the at least two insulating layers is inserted into the X-ray tube 800, the entire insulating layer group of the insulating plug 300 may be located in the housing 810 of the X-ray tube 800. In some embodiments, as shown in FIG. 9, after the insulating plug 300 including the at least two insulating layers is inserted into the X-ray tube 800, part of the insulating layer group of the insulating plug 300 may be located in the housing of the X-ray tube 800.

It should be noted that the description of X-ray tube 800 is for illustrative purposes only and is not intended to limit the scope of the present disclosure. For those having ordinary skills in the art, various variations and modifications can be made based on the present disclosure. However, these changes and modifications do not depart from the scope of the present disclosure.

FIG. 9 is a side view illustrating an exemplary socket device according to some embodiments of the present disclosure. As shown in FIG. 9, in some embodiments, the second wire 910 may be provided in the socket device 900. The second wire 910 may be configured to electrically connect the first wire 310 in the insulating plug 300 after the insulating plug 300 is connected to the socket device 910, so as to power the X-ray tube to form a DC electric field.

Taking a structure of two socket devices (the first socket device 830 and the second socket device 840) of the X-ray tube 800 as the structure of the socket device 900 for an example, the second wire (e.g., the second wire 910) in the first socket device 830 may be electrically connected to the first wire 310 in the insulating plug after the insulating plug 300 on the high-voltage cable is connected to the first socket device 830, and the high voltage generator may provide a negative voltage for the cathode 821 of the X-ray tube 800. The second wire (e.g., the second wire 910) in the second socket device 840 may be electrically connected to the first wire 310 in the insulating plug after the insulating plug 300 on the high-voltage cable is connected to the second socket device 830, and the high voltage generator may provide a positive voltage for the anode 823 of the X-ray tube 800, thereby meeting the needs of the X-ray generation system for generating X-rays.

As shown in FIG. 9, in some embodiments, the socket device 900 may also include a sealing member 920 in sealing connection with a shell (e.g., the shell 817) of the X -ray tube or a shell of the high-voltage cable. The sealing member 920 may form a cavity K configured to accommodate the insulating plug 300. In some embodiments, an insulating medium (e.g., insulating oil, insulating gas, etc.) may also be accommodated between the sealing member 920 and the insulating plug to prevent high voltage in the X-ray tube from leaking out.

In some embodiments, the sealing member 920 may also serve as a receptacle for the socket device 900. As shown in FIG. 9, a center line of the sealing member 920 may include a terminal 930. The second wire 910 of the socket device 900 may be connected to the first conductor 310 of the insulating plug 300 at the terminal 930 to realize energization of the X-ray tube. The terminal 930 may be in a disconnected state when the socket device 900 is not connected to the insulating plug 300.

It should be noted that the above description of the socket device 900 is only for example and explanation, and does not limit the scope of application of the present disclosure. For those skilled in the art, various modifications and changes can be made to the socket device 900 under the guidance of the present disclosure. However, such modifications and changes remain within the scope of the present disclosure.

FIG. 10 is a side view illustrating an exemplary socket device according to some embodiments of the present disclosure. As shown in FIG. 10, in some embodiments, a socket device 1000 may include a first wire 310 of an insulating plug, a first insulating layer 320 covering the first wire 310, and a second insulating layer 330 covering the first insulating layer 320. In some embodiments, the socket device 1000 may further include a sealing member 1010 in sealing connection with a shell (e.g., the shell 810 of the X-ray tube 800 or the shell of a high-voltage cable).

Based on the above content, in some embodiments, a volume resistivity of the second insulating layer 330 of the socket device 1000 may be greater than a volume resistivity of the first insulating layer 320. A ratio of the volume resistivity of the second insulating layer 330 to the volume resistivity of the first insulating layer 320 of the socket device 1000 may be a ratio of a radius of the second insulating layer 330 to a radius of the first insulating layer 320. In some embodiments, a first insulating connection layer may be disposed between the second insulating layer 330 and the first insulating layer 320 of the socket device 1000. In some embodiments, the socket device 1000 may further include at least one outer insulating layer (e.g., outer insulating layer 340, the outer insulating layer 350, etc.) covering the second insulating layer 330. In some embodiments, a volume resistivity of one of the at least one outer insulating layer closest to the second insulating layer may be greater than the volume resistivity of the second insulating layer 330. In some embodiments, a second insulating connection layer may be disposed between the outer insulating layer 340 and the second insulating layer 330. More descriptions may be found in the relevant descriptions of the insulating plug 300, which are not repeated here.

By arranging the socket device as a composite structure including the plurality of insulating layers, the volume resistivity of each insulating layer can increase according to the radius, which can reduce the electric field intensity of the high field intensity region and increase the electric field intensity of the low field intensity region, making the distribution of the overall electric field intensity of the insulating layer group more uniform, thereby extending the service life of the X-ray tube. In a steady-state DC electric field, the insulating layer group is not limited by the discharge time of the X-ray tube, and can still achieve uniform distribution of the electric field intensity.

It should be noted that the above description of the socket device 1000 is only for example and explanation, and does not limit the scope of application of the present disclosure. For those skilled in the art, various modifications and changes can be made to the socket device 1000 under the guidance of the present disclosure. However, such modifications and changes remain within the scope of the present disclosure.

The embodiments of the present disclosure further provide a high voltage generator including at least one socket device (e.g., the socket device 900 or the socket device 1000).

The embodiments of the present disclosure further provide a high-voltage cable. At least one end of the high-voltage cable may be provided with an insulating plug 300. For example, one end of the high-voltage cable connected to an X-ray tube or one end of the high-voltage cable connected to a high voltage generator may be provided with the insulating plug 300. As another example, one end of the high-voltage cable connected to the high voltage generator may be provided with a first insulating plug 300, and another end of the high-voltage cable connected to the X-ray tube may be provided with a second insulating plug 300. By arranging the insulating plug 300 on the at least one end of the high-voltage cable, non-uniform distribution of the electric field intensity at the connection of the high-voltage cable can be avoided, thereby avoiding insulation breakdown of the high-voltage cable, and improving the stability and safety of the high-voltage cable.

In some embodiments, at least one socket device 900 may be provided on both the high voltage generator and the X-ray tube. The insulating plugs 300 at both ends of the high-voltage cable may be respectively connected to the socket device of the high voltage generator and the socket device of the X-ray tube. An insulating plug end of the high-voltage cable connected to the socket device 900 of the high voltage generator may receive a high voltage of the high voltage generator through the first wire 310 of the insulating plug. An insulating plug end of the high-voltage cable connected to the socket device 900 of the X-ray tube may provide the high voltage for the X-ray tube through the first wire 310. By arranging the socket device 900 on the high voltage generator and the X-ray tube, the socket device 900 can be electrically connected to the high-voltage cable including the insulating plug 300, so that the distribution of the electric field intensity of the high voltage generator and the X-ray tube at the connection with the high-voltage cable can be uniform, thereby improving the safety and stability of the X-ray emission system, and extending the service life of the X-ray emission system.

The basic concept has been described above. Obviously, for those skilled in the art, the above detailed disclosure is only an example, and does not constitute a limitation to the present disclosure. Although not expressly stated here, those skilled in the art may make various modifications, improvements and corrections to the present disclosure. Such modifications, improvements and corrections are suggested in this disclosure, so such modifications, improvements and corrections still belong to the spirit and scope of the exemplary embodiments of the present disclosure.

Meanwhile, the present disclosure uses specific words to describe the embodiments of the present disclosure. For example, “one embodiment”, “an embodiment”, and/or “some embodiments” refer to a certain feature, structure or characteristic related to at least one embodiment of the present disclosure. Therefore, it should be emphasized and noted that references to “one embodiment” or “an embodiment” or “an alternative embodiment” two or more times in different places in the present disclosure do not necessarily refer to the same embodiment. In addition, certain features, structures or characteristics in one or more embodiments of the present disclosure may be properly combined.

In addition, unless clearly stated in the claims, the sequence of processing elements and sequences described in the present disclosure, the use of counts and letters, or the use of other names are not used to limit the sequence of processes and methods in the present disclosure. While the foregoing disclosure has discussed by way of various examples some embodiments of the invention that are presently believed to be useful, it should be understood that such detail is for illustrative purposes only and that the appended claims are not limited to the disclosed embodiments, but rather, the claims are intended to cover all modifications and equivalent combinations that fall within the spirit and scope of the embodiments of the present disclosure. For example, although the implementation of various components described above may be embodied in a hardware device, it may also be implemented as a software only solution, e.g., an installation on an existing server or mobile device.

In the same way, it should be noted that in order to simplify the expression disclosed in this disclosure and help the understanding of one or more embodiments of the invention, in the foregoing description of the embodiments of the present disclosure, sometimes multiple features are combined into one embodiment, drawings or descriptions thereof. This method of disclosure does not, however, imply that the subject matter of the disclosure requires more features than are recited in the claims. Rather, claimed subject matter may lie in less than all features of a single foregoing disclosed embodiment.

In some embodiments, counts describing the quantity of components and attributes are used. It should be understood that such counts used in the description of the embodiments use the modifiers “about”, “approximately” or “substantially” in some examples. Unless otherwise stated, “about”, “approximately” or “substantially” indicates that the stated figure allows for a variation of ±20%. Accordingly, in some embodiments, the numerical parameters used in the disclosure and claims are approximations that can vary depending upon the desired characteristics of individual embodiments. In some embodiments, numerical parameters should consider the specified significant digits and adopt the general digit retention method. Although the numerical ranges and parameters used in some embodiments of the present disclosure to confirm the breadth of the range are approximations, in specific embodiments, such numerical values are set as precisely as practicable.

Each of the patents, patent applications, publications of patent applications, and other material, such as articles, books, specifications, publications, documents, things, and/or the like, referenced herein is hereby incorporated herein by this reference in its entirety for all purposes, excepting any prosecution file history associated with same, any of same that is inconsistent with or in conflict with the present document, or any of same that may have a limiting affect as to the broadest scope of the claims now or later associated with the present document. By way of example, should there be any inconsistency or conflict between the description, definition, and/or the use of a term associated with any of the incorporated material and that associated with the present document, the description, definition, and/or the use of the term in the present document shall prevail.

In closing, it is to be understood that the embodiments of the application disclosed herein are illustrative of the principles of the embodiments of the application. Other modifications that may be employed may be within the scope of the application. Thus, by way of example, but not of limitation, alternative configurations of the embodiments of the application may be utilized in accordance with the teachings herein. Accordingly, embodiments of the present application are not limited to that precisely as shown and described.

What is claimed is:

1. An insulating plug, comprising a first wire and an insulating layer group covering the first wire, the insulating layer group including at least two insulating layers; wherein
the insulating layer group includes a first insulating layer covering the first wire, and a second insulating layer covering the first insulating layer; and a volume resistivity of the second insulating layer is greater than a volume resistivity of the first insulating layer.

2. The insulating plug of claim 1, wherein a ratio of the volume resistivity of the first insulating layer to the volume resistivity of the second insulating layer is a ratio of the radius of the first wire to the radius of the first insulating layer.

3. The insulating plug of claim 2, wherein electric field intensities at the inner surfaces of the at least two insulating layers are equal or approximately equal.

4. The insulating plug of claim 1, wherein the insulating layer group further includes at least one outer insulating layer covering the second insulating layer, and a volume resistivity of the at least one outer insulating layer is greater than the volume resistivity of the second insulating layer.

5. The insulating plug of claim 4, wherein a ratio of the volume resistivity of the second insulating layer to the volume resistivity of one of the at least one outer insulating layer closest to the second insulating layer is a ratio of the radius of the first insulating layer to the radius of the second insulating layer.

6. The insulating plug of claim 4, wherein
the insulating layer group further includes at least two outer insulating layers covering the second insulating layer; and
the volume resistivity of a second outer insulating layer of the at least two outer insulating layers is greater than the volume resistivity of a first outer insulating layer of the at least two outer insulating layers, the first outer insulating layer covering the second insulating layer and the second outer insulating layer covering the first outer insulating layer.

7. The insulating plug of claim 6, wherein a ratio of the volume resistivity of the first outer insulating layer to the volume resistivity of the second outer insulating layer is a ratio of the radius of the second insulating layer to the radius of the first outer insulating layer.

8. The insulating plug of claim 1, wherein a thickness of the insulating layer group is configured to be positively correlated with a maximum voltage carried by the first wire.

9. The insulating plug of claim 1, wherein under a certain voltage, an average value of an electric field intensity at the insulating plug is negatively correlated with a thickness of the insulating layer group.

10. The insulating plug of claim 1, wherein
an insulating connection layer is provided between the first insulating layer and the second insulating layer; and
the insulating connection layer includes a first surface and a second surface which are oppositely arranged, the first surface is connected to the first insulating layer, and the second surface is connected to the second insulating layer.

11. The insulating plug of claim 10, wherein a thickness of the insulating connection layer is in a range of 1 mm to 2 mm.

12. The insulating plug of claim 10, wherein a volume resistivity of the insulating connection layer is equal to or approximately equal to the volume resistivity of the first insulating layer or the second insulating layer.

13. The insulating plug of claim 1, wherein lengths of the first insulating layer and the second insulating layer are configured to be positively correlated with a maximum voltage carried by the first wire.

14. The insulating plug of claim 1, wherein lengths of the first insulating layer and the second insulating layer are the same.

15. A high-voltage cable, wherein at least one end of the high-voltage cable is equipped with an insulating plug;
the insulating plug includes a first wire and an insulating layer group covering the first wire, the insulating layer group including at least two insulating layers; wherein
the insulating layer group includes a first insulating layer covering the first wire, and a second insulating layer covering the first insulating layer; and a volume resistivity of the second insulating layer is greater than a volume resistivity of the first insulating layer.

16. An X-ray tube, comprising:
a socket device configured to connect an insulating plug, the insulating plug including a first wire and an insulating layer group covering the first wire, the insulating layer group including at least two insulating layers; wherein
the insulating layer group includes a first insulating layer covering the first wire, and a second insulating layer covering the first insulating layer; and a volume resistivity of the second insulating layer is greater than a volume resistivity of the first insulating layer; and
a second wire disposed in the socket device, wherein the second wire is configured to electrically connect the first wire in the insulating plug after the insulating plug is connected to the socket device, so as to power the X-ray tube.

17. The X-ray tube of claim 16, wherein a ratio of the volume resistivity of the first insulating layer to the volume resistivity of the second insulating layer is a ratio of the radius of the first wire to the radius of the first insulating layer.

18. The X-ray tube of claim 16, wherein the insulating layer group further includes at least one outer insulating layer covering the second insulating layer, and a volume resistivity of the at least one outer insulating layer is greater than the volume resistivity of the second insulating layer.

19. The X-ray tube of claim 16, wherein the socket device is electrically connected to at least one of a cathode or an anode of the X-ray tube.

20. The X-ray tube of claim 19, further comprising a shell configured to accommodate the cathode and the anode of the X-ray tube, wherein the socket device includes a sealing member in sealing connection with the shell, and the sealing member includes a cavity for accommodating the insulating plug.
